(19) United States
(12) Patent Application Publication
(10) Pub. No.: US 2008/0076140 A1
(43) Pub. Date: Mar. 27, 2008

(54) Biomarkers of Alzheimer's Disease

(75) Inventor: Simon Lovestone, London (GB)

Correspondence Address: BANNER & WITCOFF, LTD., 1100 13th STREET, N.W., SUITE 1200, WASHINGTON, DC, 20005-4051, US

(73) Assignee: King's College London, London, GBN (GB)

(21) Appl. No.: 11/630,769

(22) PCT Filed: Jul. 1, 2005
(86) PCT No.: PCT/GB05/02592

§ 371 Date: Oct. 5, 2007

(30) Foreign Application Priority Data
Jul. 2, 2004 (GB) 0414894.6

(51) Int. Cl. G01N 33/68

(52) U.S. Cl. 435/7.92

(57) ABSTRACT

A test for diagnosing Alzheimer's Disease comprises measuring levels of GSK-3 in cells or body fluid in a sample taken from a human subject. The test provides a relatively noninvasive method of diagnosing Alzheimer's Disease, and may be useful in predicting which individuals with mild cognitive impairment will develop Alzheimer's Disease.

This invention relates to the diagnosis of Alzheimer's disease and more particularly to reliable diagnosis at early stages of this disease.

Alzheimer's disease (AD) is a devastating condition affecting over 600,000 people in the United Kingdom alone. The progress being made in the understanding of AD and related disorders at a molecular level is not being matched by progress in clinical assessment. Diagnosis is performed by clinical interview with supplementary investigations to exclude rare treatable causes of confusion. As specific treatments are generated for prevention or modification of AD, the limitations in diagnosing the disease will become more clinically relevant and might substantially delay effective assessment and utilisation of treatments.

Progress in clinical trials of putative disease modification therapies would be much enhanced if these could be directed towards those suffering from early dementia. As there is evidence that the cognitive decline of AD and other disorders is preceded by neuropathological deterioration, which starts many years earlier, then it is probable that disease modification therapies would effectively be secondary preventative strategies. However, whilst differential diagnosis of established dementia is difficult, the reliable detection of a neurodegenerative process before the onset of a full dementia syndrome is not currently possible. There are no independent markers of the prodromal phase of dementia (even when cognition is demonstrably compromised) and it is difficult to predict which individuals with subjective memory impairment will progress to full dementia.

Measurement of change is also highly problematical. Thus, in AD and related disorders all three areas of assessment—differential diagnosis, early diagnosis and measurement of change—are mostly limited at present to clinical assessment and do not use biomarkers. The Alzheimer's Association and the National Institute of Ageing recognised the urgent need for an independent marker of disease status, and a consensus group hosted by them laid down the criteria for a successful biomarker. Ideally it would be ‘reliable, reproducible, non-invasive, simple to perform and inexpensive’ whilst having a sensitivity and specificity of more than 80%. No such marker is yet available.

AD and fronto-temporal dementia (FTD) are part of the group of disorders known as the tauopathies characterised by aggregates of highly phosphorylated tau protein. Various tau kinases have been identified of which glycogen synthase kinase-3 (GSK-3) and CDK5 appear to be the best candidates. However, although both have been shown to phosphorylate tau in vitro, in neurons and in transgenic models there is only modest evidence that either is altered in vivo. GSK-3 has been shown to co-localise with neurofibrillary tangles, and p25, the precursor of CDK5, has been shown to be present to excess in post mortem AD brain. However, such post-mortem studies pose the question as to whether these observations are a cause or an effect of neurodegeneration; an important issue carrying direct therapeutic implications.

Bhat et al. (2004; Journal of Neurochemistry 89, 1313-1317) discuss GSK-3 as a drug target for therapies of diseases of the central nervous system. They suggest that the active form of GSK-3β is increased in AD brain, although they state that increased levels of total GSK-3 have not been consistently observed in AD brain. Nevertheless, they go on to suggest that GSK-3 inhibition could be of therapeutic benefit in AD.

The use of GSK-3 inhibitors in therapy of various diseases, including AD, is also discussed by Eldar-Finkelman (2002; Trends in Molecular Medicine 8, 126-132).

However, GSK-3 has never been suggested as a suitable prognostic or diagnostic marker for AD.

Previous Attempts to Make a Laboratory Based Diagnostic Test for AD

Protein Changes in Cerebro Spinal Fluid (CSF)

Various proteins have been shown to differ in CSF of AD patients relative to aged controls. CSF quantification of tau by ELISA has been extensively examined in over 25 studies—including more than 1100 patients with AD. In these studies the amount of tau is significantly and substantially elevated in AD. Other studies of CSF in AD provide evidence that metabolic products of amyloid precursor protein (APP) are also altered in AD. Thus, soluble APP is decreased in symptomatic carriers of pathogenic APP mutations and in sporadic AD but is also low in spongiform encephalopathies. The Aβ1-42 peptide, and in some but not all studies the Aβ1-40 peptide as well, is also decreased in dementia. Thus studies in CSF have found both key proteins altered in AD (tau increasing and Aβ decreasing) and moreover many other proteins putatively linked with the pathophysiology of AD are also altered. None, however, has been shown to be sensitive to early change, nor to discriminate between AD and other neurodegenerative diseases. A more fundamental difficulty with all of these studies arises, however, in that lumbar puncture is an invasive investigation, and this will limit its use in routine investigation, either for early diagnosis or for monitoring change. Diagnostic markers in AD will only proceed to clinical utility with the generation of tests based upon analysis of serum, plasma or urine.

Protein Changes in Serum or Plasma

Other studies have investigated the possibility of protein or cellular changes in serum or plasma. Although one study identified tau immunoreactivity in serum there was no relationship to dementia. Aβ42 is reported in one study to be elevated in serum in AD although it is difficult to reconcile this finding with a corresponding decrease in CSF, and more studies are needed. Perhaps the earliest reports of peripheral markers in AD were those of changes in platelet membrane fluidity—a finding that, although not uncontroversial, continues to raise interest.

WO 2004/027429 discloses a method and diagnostic kit for diagnosing AD, among patients with MCI, by testing for the enzyme glutamine synthetase in blood. Since glutamine synthetase is astrocyte-specific, it could simply be an indicator of neuronal damage.

There is therefore a need for a new diagnostic or prognostic test for AD that involves testing of only peripheral samples.

According to a first aspect of the present invention, there is provided a method for the prognosis or diagnosis of AD comprising measuring levels of GSK-3 in cells or body fluid in a sample taken from a human subject.

An association between GSK-3 and AD has been established, and the ability to test for this enzyme means that this test is easily carried out to provide the desired diagnosis.

The total amount of GSK-3 isotypes may be measured and compared with controls. This can provide an indication of whether a subject has or is likely to develop AD.

Preferably the levels of both active and inactive GSK-3 are measured. An increase in active GSK-3 indicates a likelihood of AD, whereas levels of inactive GSK-3 remain unaltered.

Preferably a positive indication of AD is determined by detecting a 20% increase in GSK-3 protein or activity. This can indicate the presence or the likelihood of future development of AD.

A method as claimed in any preceding claim, wherein the human subject has been diagnosed with mild cognitive impairment (MCI). This test may be particularly useful in assessing whether a patient with MCI is likely to develop AD.

A method as claimed in any preceding claim, wherein the sample tested is serum or plasma. This allows a particularly simple method of testing for the presence of GSK-3, without the need for an invasive procedure.

According to a second aspect of the present invention, there is provided a kit for use in a test for the prognosis or diagnosis of Alzheimer's disease comprising at least one of the following antibodies: antibody to GSK-3 and antibody to active GSK-3.

These antibodies enable the measurement of levels of GSK-3 and/or active GSK-3.

Preferably, the antibody to GSK-3 is anti-GSK-3 α/β and the antibody to active GSK-3 is anti-GSK-3 α/β Tyr 216/219.

The kit may further comprise antibody to inactive GSK-3, which is preferably anti-GSK-3 α/β Ser 21/9.

According to a third aspect of the present invention there is provided anti-GSK-3 antibody or anti-inactive GSK-3 antibody for use in a test for the diagnosis or prognosis of Alzheimer's disease.

According to a fourth aspect of the present invention there is provided use of anti-GSK-3 antibody anti-inactive GSK-3 antibody in the manufacture of a kit for use in a test for the diagnosis or prognosis of Alzheimer's disease.

Approach to Biomarkers for AD

Being a tau kinase, it is possible that GSK-3 could be involved in the pathogenesis of AD. However, studies of GSK-3 in post-mortem AD brain have not shown consistent results and other data suggest other tau kinases such as CDK5 might be more important. Yet another school of thought suggests that the changes in tau phosphorylation are all secondary and not primary to the disease process. Levels of GSK-3 in early AD, and the possibility of markers of GSK-3 protein and activity in easily accessed material such as blood cells acting as biomarkers were investigated. There have been no previous studies of GSK-3 in peripheral samples in AD. However, GSK-3 protein and activity has been measured in peripheral samples in other conditions. Previously GSK3 has been assayed in schizophrenia with conflicting results (Nadri et al. (2002) Psychiatry Research 112, 51-57). There is no known relationship between schizophrenia and Alzheimer's disease.

In this study the total amount of GSK-3 protein (both the alpha and beta isoforms of the enzyme) and the levels of active (phosphorylated at Tyr216/219) and inactive enzyme (phosphorylated at Ser9/21) were compared. As samples crude extracts of white cells were used, and GSK-3 was analysed using standard quantitative western blotting techniques.

Practical Application

Methods

Subjects: People with AD (NINCDS-ADRDA defined probable) were identified through Old Age Psychiatry services. Normal elderly were identified through South London GP based age/sex registers. All subjects were assessed using well known systematic and validated procedures. Subjects studied had established AD (n=27), mild cognitive impairment (commonly held to be a prodrome of AD) (n=16) and normal controls (n=15).

Samples: Fresh venous blood is collected in a BD vacutainer K3E 15% tubes. The blood is spun at 3000 rpm for 8 min and the plasma is aliquoted and stored at −70° C. The remaining buffy coat is carefully collected using a P200 pipette (some red blood cells collected is acceptable). The buffy coat is transferred to a clean micro tube and stored at −20° C. until use.

Analysis: The buffy coat sample is defrosted and added to 10 ml red cell lysis buffer (10 mM Tris, 5 mM MgCl₂ and 10 mM NaCl pH 7.6) in a 15 ml Falcon tube and left on ice for 30 min. The lysed samples are then spun at 2800 rpm for 10 min and the supernatant discarded. A further 10 ml of lysis buffer is added, the tubes vortexed and then left on ice for a further 20 min.

The samples are once more spun at 2800 rpm for 10 min and the supernatant is discarded. To the pellet, 300 μl of 2× sample buffer is added and transferred from the Falcon tube to a micro tube and heated at 100° C. for 10 min.

For western blot analysis 10 μl of the lysed sample is loaded onto a 10% SDS-PAGE gel and separated at 150V for 60 min. The proteins are transferred to a substrate, which is subsequently blotted and probed overnight at 4° C. with β-actin AC-15 antibody for normalisation to total protein (Sigma), GSK-3 α/β antibody for total GSK-3 protein (Bioquote), GSK-3 α/β Ser 21/9 antibody for inactive GSK-3 and GSK-3 α/β Tyr 216/219 antibody for active GSK-3 (Signal Transduction). Bands were detected with a chemiluminescence Western detection kit (Amersham). The blots are then scanned using a Bio-Rad GS710 scanner, and the optical density of immunoreactive bands was quantified using the Bio-Rad Quantity One image analysis system.

Results

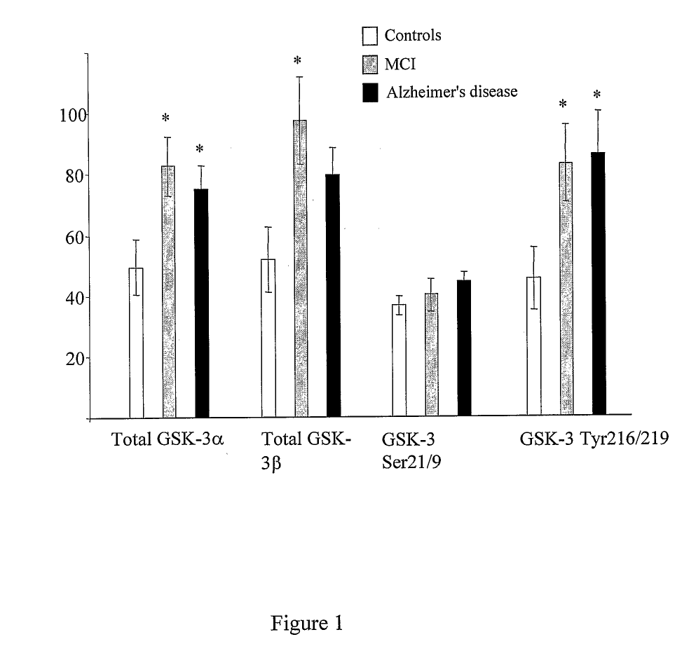

The results are illustrated in FIG. 1. GSK-3 protein (both alpha and beta isoforms) and activity (as represented by the Tyr 216/219 phospho-specific antibody) are higher in MCI and in AD than in normal elderly controls (p<0.05). Levels of inactive GSK-3 (as represented by the Ser 21/9 antibody) are unaltered. A measure of about 20% or more of GSK-3 protein or activity is an indication of the presence or likelihood of future development of AD.

### CONCLUSIONS

These data demonstrate that in circulating white cells the activity of GSK-3 is increased due both to an increase in total protein and to an increase in the active relative to the inactive enzyme, in Alzheimer's and also, importantly, in the prodromal condition of MCI.

The methods described above can be improved by developing other immunological detection methods including enzyme linked immuno assays (ELISA) for example. Such approaches lend themselves readily to clinical applications. Measurement of GSK-3 protein and activity in peripheral samples such as white cells or lymphocytes therefore provides effective diagnosis, early detection and staging of Alzheimer's disease and related dementias.

The assessment of GSK-3 protein and activity can be used as an early diagnostic marker test to distinguish people with AD from other conditions that can be confused with it (e.g. other conditions that may involve memory loss such as depression or anxiety or other disorders causing dementia, such as vascular dementia or dementia with Lewy bodies). It may also assist in determining whether a person with MCI is likely to progress to dementia, or to monitor the progression of disease in response to treatments (currently only symptomatic markers are available). This marker will therefore help in the monitoring of the effects of therapies designed for disease modification and can be used as a surrogate marker for disease modification in clinical trials. It may also help in predicting which patents with AD are most likely to respond to therapies.

1. A method for the prognosis or diagnosis of Alzheimer's disease comprising measuring levels of GSK-3 in a blood sample taken from a human subject.

2. A method as claimed in claim 1, in which the total amount of GSK-3 isotypes is measured and compared with controls.

3. A method as claimed in claim 1, wherein levels of both active and inactive GSK-3 are measured, and wherein an increase in the level of active GSK-3 relative to the level of inactive GSK-3 indicates Alzheimer's disease.

4. A method as claimed in claim 1, 2, or 3, in which a positive indication of Alzheimer's disease is determined by detecting a 20% increase in GSK-3 protein or activity.

5. A method as claimed in any of claims 1, 2, or 3, wherein the human subject has been diagnosed with mild cognitive impairment.

6. A method as claimed in any of claims 1, 2, or 3, wherein the sample tested is selected from the group consisting of: serum, plasma, white blood cells, and lymphocytes.

7. A kit for use in a test for the prognosis or diagnosis of Alzheimer's disease comprising an antibody to inactive GSK-3 and an antibody to active GSK-3.

8. A kit as claimed in claim 7, wherein and the antibody to active GSK-3 is anti-GSK-3 α/β Tyr 216/219.

9. (canceled)

10. A kit as claimed in claim 7 or 8, wherein the antibody to inactive GSK-3 is anti-GSK-3 α/β Ser 21/9.

11-12. (canceled)

13. A method according to claim 3, wherein an increase in the level of active GSK-3 when the level of inactive GSK-3 remains unaltered indicates Alzheimer's disease.

14. The method of claim 13, in which a positive indication of Alzheimer's disease is determined by detecting a 20% increase in GSK-3 protein or activity.

15. The method of claim 13, wherein the human subject has been diagnosed with mild cognitive impairment.

16. The method of claim 13, wherein the sample tested is selected from the group consisting of: serum, plasma, white blood cells, and lymphocytes.
